(19) United States
(12) Patent Application Publication
(10) Pub. No.: US 2020/0003588 A1
(43) Pub. Date: Jan. 2, 2020

(54) OPTICAL FIBER SENSING FOR HIGHWAY MAINTENANCE

(71) Applicant: NEC Laboratories America, Inc., Princeton, NJ (US)

(72) Inventors: Ming-Fang HUANG, PRINCETON, NJ (US); Ting WANG, WEST WINDSOR, NJ (US)

(73) Assignee: NEC LABORATORIES AMERICA, INC, PRINCETON, NJ (US)

(21) Appl. No.: 16/454,053

(22) Filed: Jun. 27, 2019

Related U.S. Application Data
(60) Provisional application No. 62/691,140, filed on Jun. 28, 2018.

(51) Int. Cl. G01D 5/26; G01N 33/42; G06N 3/08

(52) U.S. Cl. CPC G01D 5/268; G06N 3/08; G01N 33/42

(57) ABSTRACT

Aspects of the present disclosure describe systems, methods and structures employing optical fiber sensing to monitor highway/roadway/street conditions (i.e., potholes, pavement cracks, etc.) in real-time, continuously, and while the highway/roadway/street remains in operation (in-service monitoring). Systems, methods, and structures according to aspects of the present disclosure may employ machine learning (ML) algorithms including neural networks to provide and or report on highway conditions so monitored/sensed. Of further advantage, systems, methods, and structures for optical fiber sensing for highway maintenance may operate in real-time, continuously, long-term, in-service, and may employ existing telecommunications optical cables without additional deployment cost(s) or disruption of telecommunications traffic.

### CROSS REFERENCE TO RELATED APPLICATIONS

This application claims the benefit of U.S. Provisional Patent Application Ser. No. 62/691,140 filed 28 Jun. 2018 the entire contents of which is incorporated by reference as if set forth at length herein.

### TECHNICAL FIELD

This disclosure relates generally to optical fiber sensing systems, methods, and structures. More particularly, it describes optical fiber sensing for highway monitoring and maintenance.

### BACKGROUND

As is known by contemporary drivers of automobiles, trucks, and other vehicles, highways, roadways, and streets oftentimes exhibit deteriorating conditions. Such conditions may deteriorate even further as the rate of vehicle traffic continues to increase and federal, state, and local governments find they are unable to adequately fund road repairs. With vehicle traffic growth rates increasing, wear and tear on streets, roads, and highways is expected to increase the cost of needed highway repairs.

When needed repairs go undetected and/or uncorrected, innumerable costs result in the form of vehicle damage, accidents, and fuel consumption—among other costs. Additionally, the longer needed repairs go unmet, such costs will continue to rise.

Given these and other considerations—systems, methods, and structures that facilitate the identification of deteriorating locations in highways, roadways, and, streets, would allow prioritization of repair efforts and would represent a welcome addition to the art.

### SUMMARY

An advance in the art is made according to aspects of the present disclosure directed to systems, methods, and structures employing optical fiber sensing to monitor highway/roadway/street conditions (i.e., potholes, pavement cracks, etc.) in real-time, continuously, and while the highway/roadway/street remains in operation (in-service monitoring).

As we shall show and describe, systems, methods, and structures according to the present disclosure may advantageously include machine learning (ML) algorithms and neural networks for classification of and subsequent determination of highway conditions that in turn may be reported for prioritization/maintenance and/or public notification via Internet and/or mobile technologies.

As used herein, the terms “highway”, “roadway”, “street”, etc., are generally used interchangeably as providing a facility or surface for vehicular traffic. They are not meant to be limiting or indicative of size in this disclosure. Similarly, “pavement” is used herein is not indicative of any specific material or its physical characteristics other than identifying a material with which something is paved.

### BRIEF DESCRIPTION OF THE DRAWING

A more complete understanding of the present disclosure may be realized by reference to the accompanying drawing in which:

FIG. 1 is a schematic diagram illustrating a smart road condition monitoring system employing optical fiber sensing according to aspects of the present disclosure;

FIG. 2 is a plot illustrative of detected/received vibration signals \ according to aspects of the present disclosure;

FIG. 3(A) is a schematic diagram illustrating a health classification for a highway/roadway pavement according to aspects of the present disclosure;

FIG. 3(B) is a plot illustrating a spectra at various frequencies indicative of pavement health according to aspects of the present disclosure; and

FIG. 4 is a flow diagram illustrating an operation of a system/method according to aspects of the present disclosure.

The illustrative embodiments are described more fully by the Figures and detailed description. Embodiments according to this disclosure may, however, be embodied in various forms and are not limited to specific or illustrative embodiments described in the drawing and detailed description.

### DESCRIPTION

The following merely illustrates the principles of the disclosure. It will thus be appreciated that those skilled in the art will be able to devise various arrangements which, although not explicitly described or shown herein, embody the principles of the disclosure and are included within its spirit and scope.

Furthermore, all examples and conditional language recited herein are intended to be only for pedagogical purposes to aid the reader in understanding the principles of the disclosure and the concepts contributed by the inventor(s) to furthering the art and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that any block diagrams herein represent conceptual views of illustrative circuitry embodying the principles of the disclosure.

Unless otherwise explicitly specified herein, the FIGs comprising the drawing are not drawn to scale.

By way of some additional background, we begin by noting that highway maintenance is a continuous, never-ending task—or set of tasks—that requires inspection, detection, and subsequent remediation—where required. Historically, inspection may have involved workers walking along the highways and making notes of conditions that require repair. Such notes may have been later entered into a database for access by different (or sometimes the same) workers to identify those conditions and locations thereof for repair.

More recently, imaging or other systems/techniques—including 2D LiDAR, hyperspectral imagery, accelerometers, ultrasonic sensors, pressure sensors and others—oftentimes attached to vehicles—to provide indications of highway conditions. Of course, such techniques fail to generally provide continuous monitoring of individual highway locations as the vehicle(s) employed are moving.

In sharp contrast—and according to aspects of the present disclosure—highways are continuously monitored, in-service, by employing optical fiber sensing. In one illustrative embodiment, such optical fiber sensing may employ telecommunications optical fiber that—in addition to carrying telecommunications traffic—is also providing sensory capability of multiple elements including vibration and frequency(ies) simultaneously. Integration of machine learning (ML) techniques including neural networks and other intelligent analyzers allow the sensing/detecting/evaluation of highway conditions such as size(s) of potholes to be performed in real-time, continuously, while live vehicular traffic is maintained (in-service). Such optical fiber sensing/detecting according to the present disclosure may subsequently initiate reporting, decision making, repair dispatching as well.

As those skilled in the art will now begin to understand and appreciate, systems, methods, and structures according to the present disclosure employing fiber-based technologies include both distributed acoustic sensing (DAS), distributed vibration sensing (DVS), distributed temperature sensing (DTS) and any combination thereof. Of particular advantage—systems, methods, and structures according to the present disclosure may advantageously employ machine learning-based intelligent analysis and analyzers to provide “smart” road condition monitoring via optical fiber cables laid (installed) underneath, alongside, or otherwise proximate to the roadway. As we shall describe further, systems, methods, and structures according to aspects of the present disclosure provide real-time, continuous, remote, in-service, technician-free solutions to difficult, highway maintenance problems.

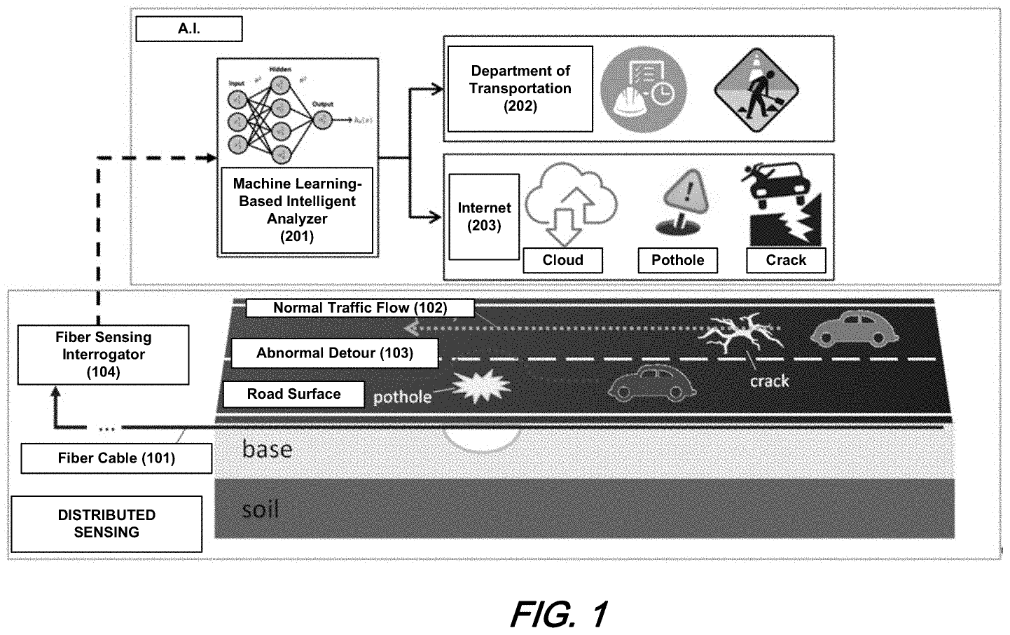

Turning now to FIG. 1, there is shown a schematic diagram illustrating a smart road condition monitoring system employing optical fiber sensing according to aspects of the present disclosure. As may be observed from that figure, the system includes a distributed sensing function/structures (DISTRIBUTED SENSING in figure) and an artificial intelligence/analysis function/structures (A.I. in figure). Conveniently, it is useful to discuss such systems with respect to these two functions/structures namely sensing and analyzing.

With reference to that figure it may be observed that shown therein is a roadway including a surface having both normal and abnormal characteristics including potholes and/or cracks in pavement. Generally, such a roadway is formed upon a base which in turn may overlie a soil. We note that such arrangement is shown only illustratively, and that different roadway construction arrangements may be made as known in the art and particular environmental requirements dictate.

Shown further in that figure, an optical fiber cable 101, is positioned proximate to the roadway and may be alongside, underneath or another location or combination thereof sufficiently proximate for our sensing purposes. More particularly, the technologies employed with the optical fiber may include DVS, DAS, and/or DTS—of combinations thereof. A sensing transmitter/receiver (transceiver) is/are located in a fiber sending interrogator 104 which is in optical communication with the optical fiber cable 101.

As will be readily appreciated by those skilled in the art, DTS may be provided by integrated temperature sensors or a common temperature sensing system/station located at a distance and providing temperature data/information via the optical fiber cable.

Traffic flow(s) and road condition(s) may be advantageously monitored via DVS and DAS technologies using the optical fiber cable. More particularly, vibration and/or frequency signals resulting from vehicular traffic on the roadway are conveyed via the optical fiber to a fiber sensing interrogator 104, which senses and initially may interpret the signals so conveyed.

As will be readily appreciated by those skilled in the art, the optical fiber may advantageously be an existing telecommunications optical fiber that is positioned sufficiently proximate to the roadway, or a newly deployed optical fiber (cable).

As noted, the technologies employed may include DVS, DAS, and DTS and sensing transmitter(s)/receiver(s) may be located in the fiber sensing interrogator that may be located proximate to—or remote from the actual roadway surface as deployment considerations dictate. As such, comprehensive, continuous, in-service, remote monitoring of the roadway is made possible by systems, methods, and structures according to aspects of the present disclosure.

Sensing data that is generated by the fiber sensing interrogator may be analyzed by an artificial intelligence (A.I.) function(s) that likewise may reside remote from the interrogator and further remote from the distributed sensing and roadway—as desired. As presently constituted, the A.I. systems include machine learning based intelligent analyzer(s) 201 and communications system(s) that provide real-time, continuous roadway conditions to—for example—an enterprise or agency or other group/individual that is charged with highway monitoring and/or maintenance 202. In addition, such analyzed data may be provided to the general public—or others—via an Internet 203 including cloud services that may identify locations/existence of potholes, cracks, etc., in pavement and roadways constructed therefrom. As will be readily understood and appreciated, such online system(s) may advantageously provide real-time and/or online reporting of highway conditions to—for example—department of transportation 202, or drivers via mobile technologies to ensure a better—and safer—driving experience.

Those skilled in the art will appreciate that two of the most significant environmental factors affecting roadway (pavement) performance are temperature and moisture content. Currently, surface temperatures of roadway pavement is estimated by a nearby weather station which may be several kilometers away from the roadway surface of interest and for which the temperature estimate is made. As will be appreciated, systems, methods, and structures according to the present disclosure may provide more accurate and localized roadway surface temperature(s) based on underground DTS techniques. Likewise, traffic flow(s) and road condition(s) may be monitored by DVS and DAS technologies.

Operationally, vibration signals are generated by a vehicle operating on/along the roadway including any cracks and/or potholes or combinations thereof. By comparing received signals associated with smooth/normal/undamaged roadway pavement with those associated with damaged roadway pavement, conditions of the roadway—and possibly their locations—may be accurately determined.

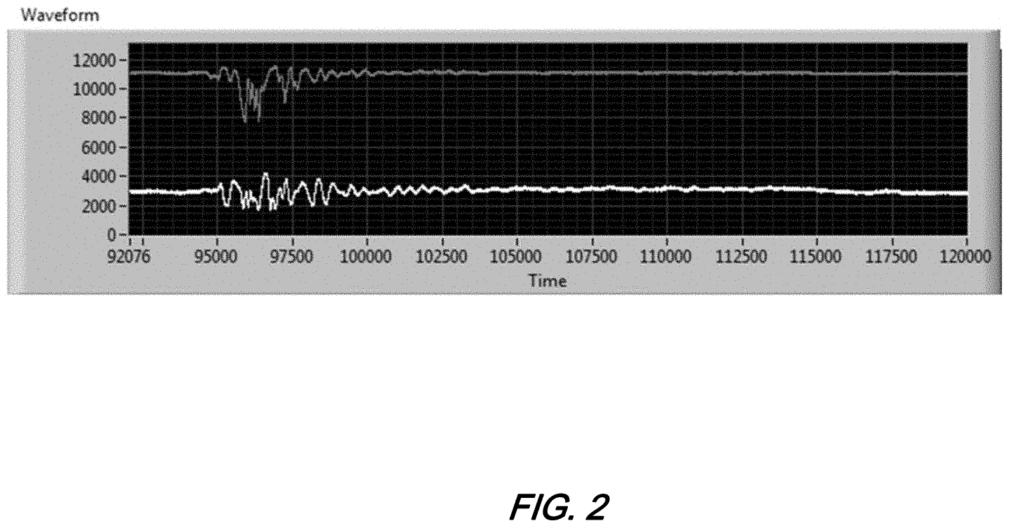

Of particular interest, different/various vibrational patterns may be associated with different roadway conditions such as the pavement crack or potholes as shown illustratively in the graph of FIG. 2. As may be observed from FIG. 2, a plot illustrative of detected/received vibration signals \ according to aspects of the present disclosure is shown.

Additionally, traffic flow (normal) patterns may be determined 102 and differentiated from abnormal flow patterns such as those resulting from a detour around a fault in the roadway 103. Long term traffic flow including traffic count(s) may be made by systems, methods, and structures according to the present disclosure thereby supporting decision making including budgeting and construction plans as well as specific roadway construction details including highway thickness and/or layers—among other physical construction characteristics of the roadway itself.

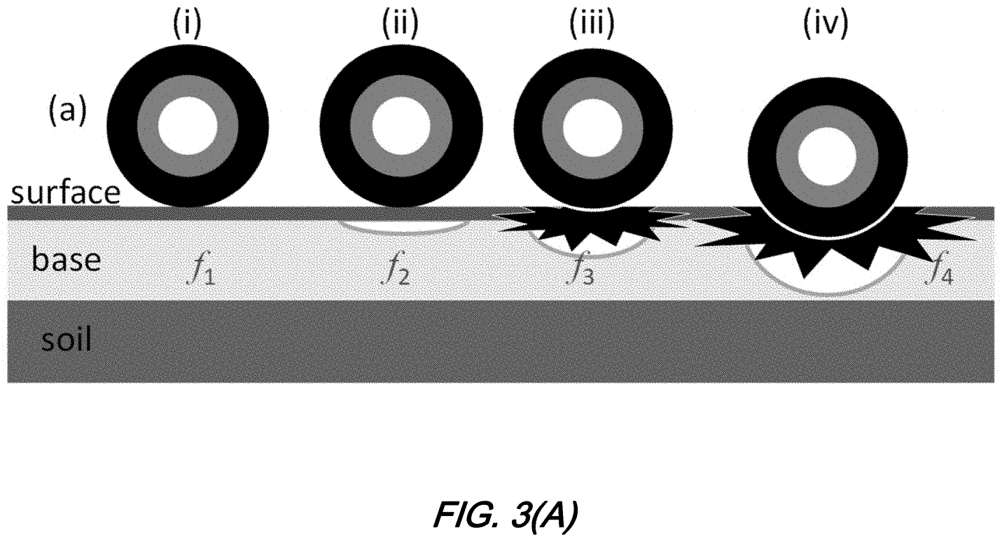

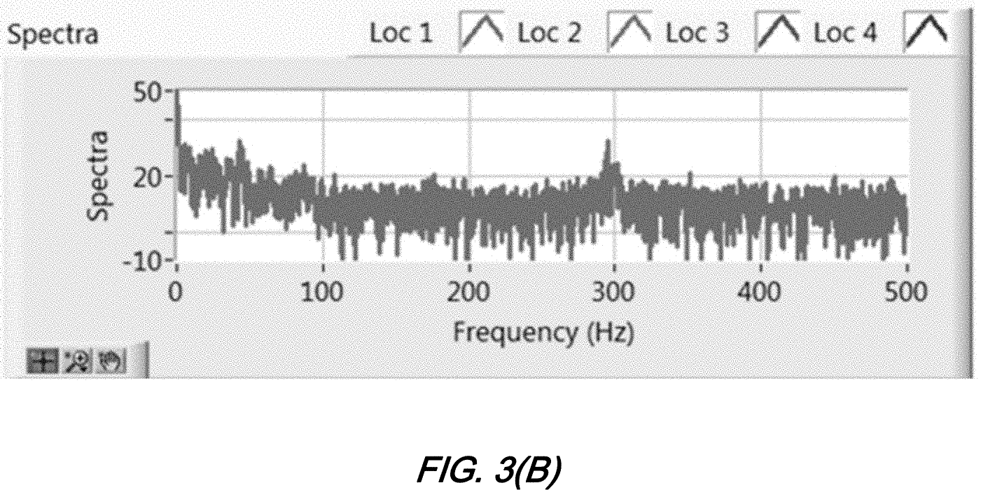

For roadway pavement health classification and determination, DAS technologies may be employed as shown in FIG. 3(A), and FIG. 3(B). FIG. 3(A) is a schematic diagram illustrating a health classification for a highway/roadway pavement according to aspects of the present disclosure. FIG. 3(B) is a plot illustrating a spectra at various frequencies indicative of pavement health according to aspects of the present disclosure.

FIG. 3(A) illustratively exhibits four (4) phases of potholes as a vehicle (indicated by a tire overrolling the roadway surface). More specifically, as a vehicle travels over a highway surface as in (i), the frequency(ies) produced f₁ is determined to be indicative of a healthy roadway pavement surface. Similarly, as a vehicle travels over the highway surface as in (ii), the frequency(ies) produced f₂ by vehicular traffic are determined to be indicative of a damaged roadway pavement surface that may—for example—have been inundated by water, rain, snow that now underlies the roadway surface possibly creating voids underneath that surface. With respect to (iii), the frequency(ies) produced f₃ by vehicular traffic are determined to be indicative of a damaged roadway pavement surface—one that could possibly cause further damage to the roadway itself or possibly the vehicle(s). Finally, with respect to (iv), the frequency(ies) produced f₄ are determined to be indicative of a more severely damaged roadway pavement surface that could very well lead to vehicle damage if the damaged roadway were used by vehicles.

As will be understood and appreciated by those skilled in the art, such roadway conditions generally become more severe and/or serious requiring more immediate attention as one progresses from condition (i) to condition (iv) as shown schematically and illustratively in the figure. As such, if maintenance is performed at condition (i), then a less expensive—less acute—repair may be made before significant structural damage occurs both to the roadway and any vehicles traveling along/upon the roadway. As shown in the figure, the pothole—in this example—produces vibrational frequencies which may be detected and by distributed acoustic sensing and an overall assessment of pavement/highway health may be determined and classified. FIG. 3(B) is a plot showing illustrative frequency response(s) for an illustrative highway having an initial condition (i) as shown in the figure.

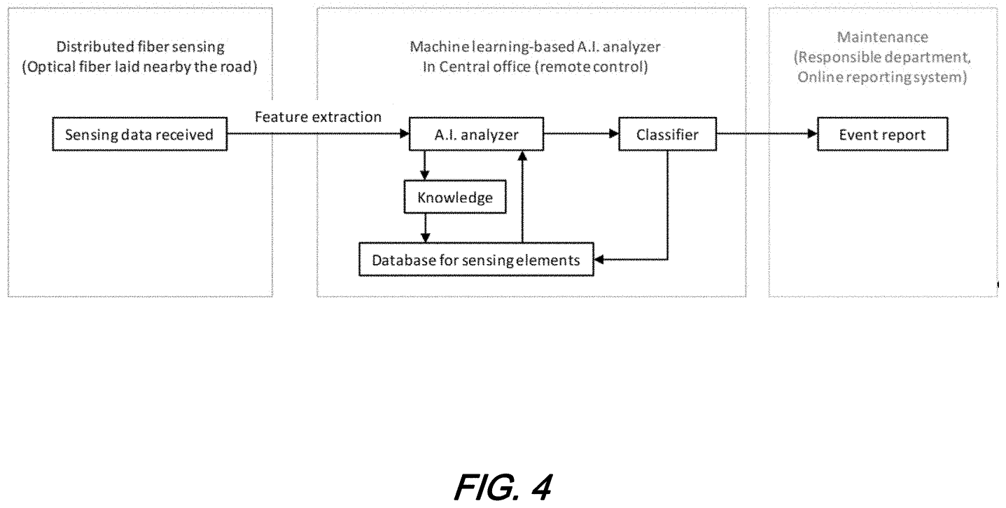

FIG. 4 is a flow diagram illustrating an operation of a system/method according to aspects of the present disclosure. Operationally, it may now be understood by those skilled in the art that sensing data is collected along a length of the fiber—or its entire length. The fiber is positioned underneath or along the roadway sufficiently proximate to provide sensory data pertaining to roadway health and/or condition(s). The data may be provided to a central office for analysis in both real-time and continuous. Based upon comparisons made with data collected, a neural network including feature extraction may be classified such that subsequent roadway health conditions may be determined from sensory data so acquired.

At this point, while we have presented this disclosure using some specific examples, those skilled in the art will recognize that our teachings are not so limited. Accordingly, this disclosure should be only limited by the scope of the claims attached hereto.

1. An optical fiber sensing system for highway maintenance comprising:
an optical fiber positioned proximate to the highway;
a plurality of sensors positioned along, and in optical communication with the optical fiber, said plurality of sensors operative continuously to respond to mechanical vibrations resulting from vehicular traffic, and produce optical signals indicative of the vibrations, said vibrational signals indicative of highway and/or traffic condition and applied to the optical fiber;
a fiber sensing interrogator in optical communication with the optical fiber, said interrogator interrogates signals produced by the plurality of sensors and produces data indicative of those signals;
a machine learning base intelligent analyzer which analyzes the data produced by the interrogator, determines highway condition and reports the highway condition so determined.

2. The optical fiber sensing system for highway maintenance according to claim 1 wherein the plurality of sensors includes one or more sensors selected from the group consisting of distributed vibrational sensors, distributed acoustic sensors, and distributed temperature sensors.

3. The optical fiber sensing system for highway maintenance according to claim 2 wherein the distributed temperature sensors provide highway surface temperatures.

4. The optical fiber sensing system for highway maintenance according to claim 2 wherein the distributed vibration sensors provide one or more of traffic count and highway surface crack/pothole indications.

5. The optical fiber sensing system for highway maintenance according to claim 2 wherein the distributed acoustic sensors provide highway pavement health indications.

6. The optical fiber sensing system for highway maintenance according to claim 2 wherein the reporting is provided to a maintenance operation.

7. The optical fiber sensing system for highway maintenance according to claim 2 wherein the reporting is provided to a public internet.

8. The optical fiber sensing system for highway maintenance according to claim 2 further comprising:
a neural network which classifies data provided by the interrogator.

9. The optical fiber sensing system according to claim 8 wherein the classified data includes pavement crack and pothole classification and their localization.

10. The optical fiber sensing system according to claim 1 wherein the optical fiber transports telecommunications data independent of and unrelated to the sensor signals.
